(19) United States
(12) Patent Application Publication
(10) Pub. No.: US 2025/0135105 A1
(43) Pub. Date: May 1, 2025

(54) FLOW MEASUREMENT SYSTEMS AND METHODS FOR MEASURING FLOW RATES OF CANCER TREATMENT SYSTEMS

(71) Applicants: Boston Scientific Scimed, Inc., Maple Grove, MN (US); Boston Scientific Medical Device Limited, Galway (IE)

(72) Inventors: Mihir Sukhatme, Mumbai (IN); Nachiket Gole, Pune (IN); Nitesh Ghananil Baviskar, Kalyan West (IN); Bruce Fullerton, Hudson, WI (US); Anthony Gedwillo, Minneapolis, MN (US); Randall Maas, Chaska, MN (US)

(21) Appl. No.: 18/930,656

(22) Filed: Oct. 29, 2024

Related U.S. Application Data
(60) Provisional application No. 63/546,345, filed on Oct. 30, 2023.

(51) Int. Cl. A61M 5/145; A61M 5/178

(52) U.S. Cl. CPC A61M 5/1452; A61M 5/178; A61M 2202/0007; A61M 2202/04; A61M 2205/3306; A61M 2205/3317; A61M 2205/3327; A61M 2205/3334; A61M 2205/502; A61M 2205/584

(57) ABSTRACT

Embodiments herein relate to flow rate measurement systems with features to indicate and/or measure flow rate therefrom. In an embodiment, a flow rate measurement device is included having a base receiver, a plunger holder, and a movement sensor. The base receiver is configured to engage with a barrel of a fluid delivery device. The plunger holder is configured to engage with a plunger of the fluid delivery device. The movement sensor is configured to detect a movement speed of the plunger holder relative to the base receiver. The flow rate measurement device can be configured to generate a signal related to the movement speed of the plunger holder relative to the base receiver. Other embodiments are also included herein.

This application claims the benefit of U.S. Provisional Application No. 63/546,345, filed Oct. 30, 2023, the content of which is herein incorporated by reference in its entirety.

### FIELD

Embodiments herein relate to flow rate measurement systems for use with cancer treatment systems and related methods.

### BACKGROUND

According to the American Cancer Society, cancer accounts for nearly 25% of the deaths that occur in the United States each year. Cancerous tumors can form if one normal cell in any part of the body mutates and then begins to grow and multiply too much and too quickly. Cancerous tumors can be a result of a genetic mutation to the cellular DNA or RNA that arises during cell division, an external stimulus such as ionizing or non-ionizing radiation, exposure to a carcinogen, or a result of a hereditary gene mutation. Regardless of the etiology, many cancerous tumors are the result of unchecked rapid cellular division. Surgery is a common first-line therapy for many cancerous tumors. However, not every tumor can be surgically removed.

### SUMMARY

Embodiments herein relate to flow rate measurement system with features to indicate and/or measure flow rate therefrom. In a first aspect, a flow rate measurement device for cancer therapy can be included having a base receiver and a plunger holder. The base receiver can be configured to engage with a barrel of a fluid delivery device, such as a syringe. The plunger holder can be configured to engage with a plunger (or similar structure) of the fluid delivery device. In this manner, as the plunger moves, the plunger holder also moves. The device can also include a movement sensor. The movement sensor can be configured to detect a movement related parameter (such as a movement speed) of the plunger holder relative to the base receiver. In this manner, the speed of depression of the plunger can be sensed. The flow rate measurement device for cancer therapy can be configured to generate a signal related to the movement speed of the plunger holder relative to the base receiver.

In a second aspect, in addition to one or more of the preceding or following aspects, or in the alternative to some aspects, the flow rate measurement device for cancer therapy can be configured to calculate a fluid flow rate based on a movement speed of the plunger holder.

In a third aspect, in addition to one or more of the preceding or following aspects, or in the alternative to some aspects, the flow rate measurement device for cancer therapy can further include a control circuit. The control circuit can be configured to receive a signal from the movement sensor and calculate a fluid flow rate based on the same.

In a fourth aspect, in addition to one or more of the preceding or following aspects, or in the alternative to some aspects, the fluid delivery device can include a syringe.

In a fifth aspect, in addition to one or more of the preceding or following aspects, or in the alternative to some aspects, the movement sensor can include a linear position sensor.

In a sixth aspect, in addition to one or more of the preceding or following aspects, or in the alternative to some aspects, the linear position sensor can include at least one selected from the group consisting of a linear encoder, an inductive position sensor, a linear variable differential transformer (LVDT), an ultrasonic position sensor, a laser position sensor, a Hall Effect sensor, a linear potentiometer, a magnetic linear position sensor, and a magnetostrictive linear position sensor.

In a seventh aspect, in addition to one or more of the preceding or following aspects, or in the alternative to some aspects, the base receiver can include a U-shaped portion.

In an eighth aspect, in addition to one or more of the preceding or following aspects, or in the alternative to some aspects, the U-shaped portion can be configured to at least partially engage with the barrel of the fluid delivery device.

In a ninth aspect, in addition to one or more of the preceding or following aspects, or in the alternative to some aspects, the base receiver can define a side slot. The side slot can be configured to allow insertion of a fluid delivery device barrel connected to a fluid tube into the base receiver.

In a tenth aspect, in addition to one or more of the preceding or following aspects, or in the alternative to some aspects, the base receiver can define a flange receiving slot. The flange receiving slot can be configured to receive a flange of the fluid delivery device arranged near a proximal end of the barrel.

In an eleventh aspect, in addition to one or more of the preceding or following aspects, or in the alternative to some aspects, the base receiver can be configured to retain a barrel of a fluid delivery device via a snap-fit or pressure-fit mechanism.

In a twelfth aspect, in addition to one or more of the preceding or following aspects, or in the alternative to some aspects, the flow rate measurement device for cancer therapy can further include a display screen. The display screen can be configured to display information related to the movement speed of the plunger holder relative to the base receiver and/or a calculated fluid flow rate.

In a thirteenth aspect, in addition to one or more of the preceding or following aspects, or in the alternative to some aspects, the display screen can be configured to display a particular color if a calculated fluid flow rate falls within a predetermined range.

In a fourteenth aspect, in addition to one or more of the preceding or following aspects, or in the alternative to some aspects, the plunger holder can overlap with a proximal end of the plunger of the fluid delivery device.

In a fifteenth aspect, a method of measuring fluid flow rate of a cancer therapy system can be included. The method can include engaging a base receiver with a barrel of a fluid delivery device. The method can further include engaging a plunger holder with a plunger of the fluid delivery device. The method can further include detecting a movement speed of the plunger holder relative to the base receiver.

In a sixteenth aspect, in addition to one or more of the preceding or following aspects, or in the alternative to some aspects, the method can further include calculating a fluid flow rate based on a movement speed of the plunger holder.

In a seventeenth aspect, in addition to one or more of the preceding or following aspects, or in the alternative to some aspects, the method can further include displaying the calculated fluid flow rate for a system user.

In an eighteenth aspect, in addition to one or more of the preceding or following aspects, or in the alternative to some aspects, the method can further include generating a signal related to the movement speed of the plunger holder relative to the base receiver.

In a nineteenth aspect, in addition to one or more of the preceding or following aspects, or in the alternative to some aspects, the step of detecting a movement speed of the plunger holder relative to the base receiver can be performed using a movement sensor.

In a twentieth aspect, in addition to one or more of the preceding or following aspects, or in the alternative to some aspects, the fluid delivery device can include a syringe.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE FIGURES

Aspects may be more completely understood in connection with the following figures (FIGS.), in which:

FIG. 1 is a diagram of a cancer treatment system in accordance with various embodiments herein.

FIG. 2 is a perspective view of a flow rate measurement device in accordance with various embodiments herein.

FIG. 3 is a side elevation view of a flow rate measurement device in accordance with various embodiments herein.

FIG. 4 is a schematic sectional view of a portion of the flow rate measurement device of FIG. 3.

FIG. 5 is a top plan view of a flow rate measurement device in accordance with various embodiments herein.

FIG. 6 is a perspective view of a fluid delivery device being inserted into a flow rate measurement device is shown in accordance with various embodiments herein.

FIG. 7 is a perspective view of a fluid delivery device engaged with a flow rate measurement device in accordance with various embodiments herein.

FIG. 8 is a flow chart of operations of a method in accordance with various embodiments herein.

While embodiments are susceptible to various modifications and alternative forms, specifics thereof have been shown by way of example and drawings, and will be described in detail. It should be understood, however, that the scope herein is not limited to the particular aspects described. On the contrary, the intention is to cover modifications, equivalents, and alternatives falling within the spirit and scope herein.

### DETAILED DESCRIPTION

As referenced above, surgery is a common first-line therapy for many cancerous tumors. However, not every tumor can be surgically removed. Another approach to treating some types of cancer can include delivering radiation to cancerous tissue to destroy cancerous cells therein. In one approach, microspheres such as small glass microspheres that have Y-90 mixed into the glass can be deposited in or near a cancerous tumor and beta radiation emitting therefrom can damage DNA of the cancerous cells inducing apoptosis and cell cycle arrest.

Delivery of microspheres can be performed using a catheter, such as a microcatheter placed in vasculature leading to a tumor, such as an artery supplying blood to the tumor. The microspheres can be combined with a fluid, such as saline, to form a suspension that is then directed through the catheter to the site of deposition.

In some approaches, a clinician must utilize a syringe or similar device to deliver the fluid to be mixed with the microspheres. The clinician advances the plunger of the syringe at a desired pressure/speed resulting in a particular flow rate of fluid through the system for a particular amount of time to a achieve a target volume of suspension delivered out of the catheter and into the patient. However, it is tedious for system users to manage aspects of this process. For example, it is difficult for system users to accurately determine the actual flow rate achieved (such as between 5 ml/min and 20 ml/min, or within other flow rate bounds).

Systems and methods herein can be used to accurately measure flow rates of fluid from fluid delivery devices. Such flow rates can be measured without making direct contact with fluids so that sterility can be maintained. In some embodiments, a flow rate measurement device for cancer therapy is included having a base receiver, a plunger holder, and a movement sensor. A fluid delivery device, such as a syringe, can engage with the flow rate measurement device. For example, the plunger of the fluid delivery device can engage with the plunger holder of the flow rate measurement device and the barrel of the fluid delivery device can engage with a portion of the base receiver of the flow rate measurement device. The plunger holder of the flow rate measurement device can be depressed and the movement sensor of the flow rate measurement device can detect a movement speed of the plunger holder relative to the base receiver and generate a signal related to the movement speed. That signal can then be converted into a fluid flow rate. In some cases, the fluid flow rate can be displayed on a display screen of the fluid flow measurement device. Thus, a clinician or device operator can observe the fluid flow rate in real time and adjust the depression speed of the plunger/plunger holder as desired.

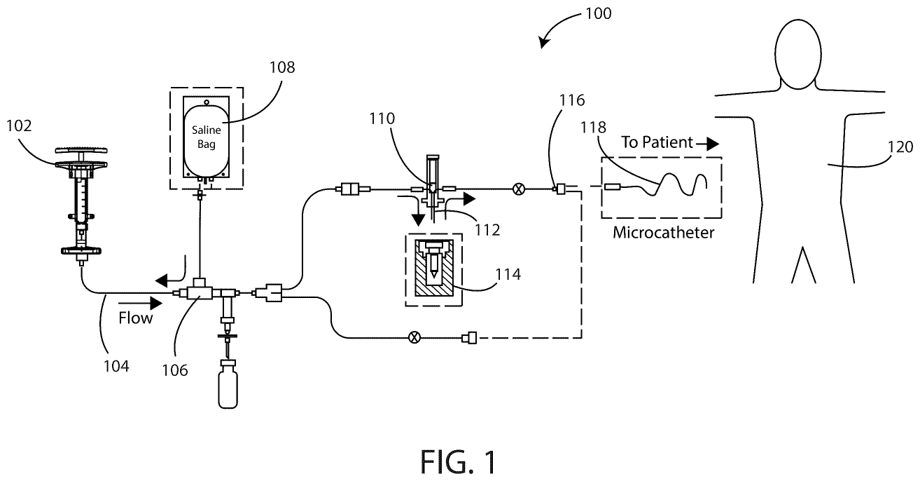

Referring now to FIG. 1, a diagram of a cancer treatment system 100 is shown in accordance with various embodiments herein. Major parts of the cancer treatment system 100 include a therapeutic fluid delivery device 102, engaged with a flow rate measurement device described in more detail below, a fluid supply tube 104, and a flow control valve 106. The cancer treatment system 100 also includes a saline supply reservoir 108. The cancer treatment system 100 also includes a fluid injection and suspension withdrawal device 110. The cancer treatment system 100 also includes a radioactive microsphere supply reservoir 114. The cancer treatment system 100 also includes an outflow port 116. The cancer treatment system 100 also includes a microcatheter 118. FIG. 1 also shows a patient 120 in which the microcatheter 118 can be inserted to deliver the therapeutic suspension of microspheres.

In use, (omitting some possible operations for ease of explanation) the clinician or other system user can pull back on the plunger holder/plunger or similar mechanism of therapeutic fluid delivery device 102 causing fluid (such as saline) to be withdrawn from the saline supply reservoir 108, through the flow control valve 106 and the fluid supply tube 104, and into the fluid delivery device 102. Then the clinician or other system user can depress the plunger holder/plunger causing fluid to flow from the therapeutic fluid delivery device 102, through the fluid supply tube 104, through the flow control valve 106, and into the fluid injection and suspension withdrawal device 110. The fluid injection and suspension withdrawal device 110 can be in fluid communication with the radioactive microsphere supply reservoir 114 and can direct a flow of fluid into the radioactive microsphere supply reservoir 114 coming from the therapeutic fluid delivery device 102 such as through one of a pair of needles, cannulas, or tubes 112. The fluid can become mixed with microspheres in the radioactive microsphere supply reservoir 114 forming a suspension which can then exit via the fluid injection and suspension withdrawal device 110 via another needle, cannula, or tube 112 and through tubing and out of the outflow port 116 and into the microcatheter 118 and into a desired site of the patient 120. It will be appreciated that various other operations can also be performed including, but not limited to, system priming, bubble removal, one or more flushing operations, and the like.

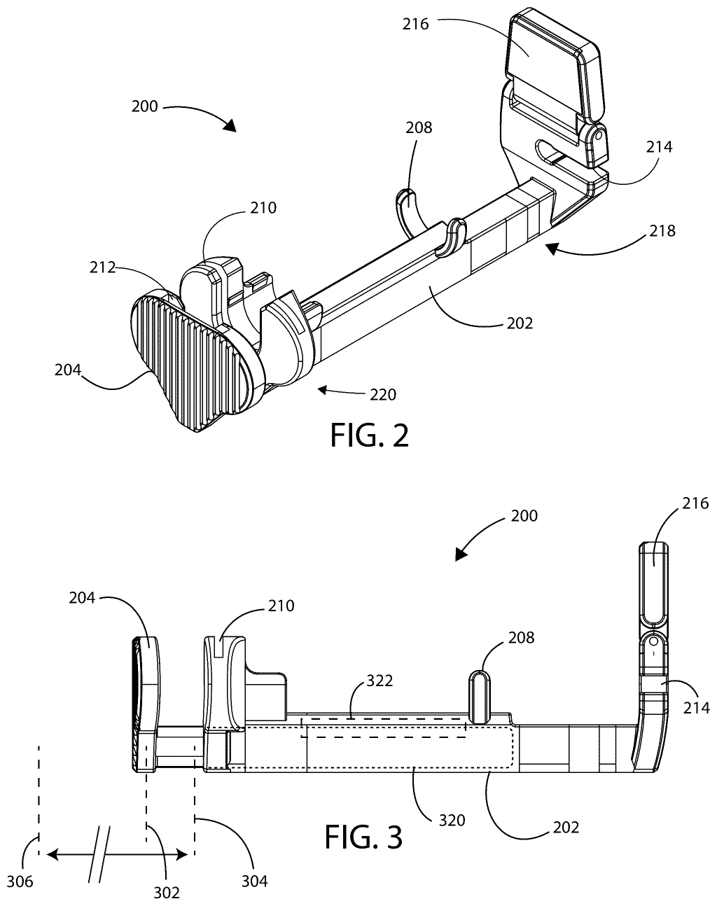

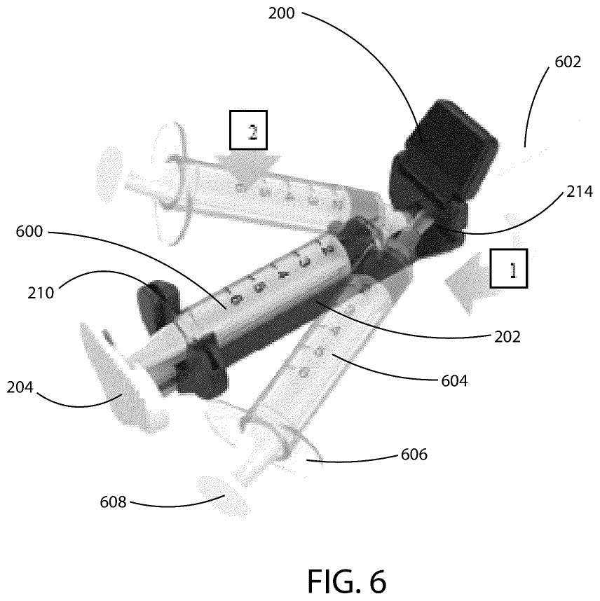

Referring now to FIG. 2, a perspective view of a flow rate measurement device is shown in accordance with various embodiments herein. In this example, the flow rate measurement device 200 includes a base receiver 202, a plunger holder 204, and a movement sensor (not shown in this view). The base receiver 202 and the plunger holder 204 can be formed of various materials including, but not limited to, polymers, composites, metals, glasses, and the like. The base receiver 202 can be configured to engage with a barrel of a fluid delivery device, described in more detail below with respect to FIG. 6. In some embodiments, the base receiver 202 can include a U-shaped portion 208 to engage with the barrel or another part of the fluid delivery device. However, many different mechanisms can be used to engage with the barrel or other part of the fluid delivery device. In some embodiments, a V-shaped portion or a square-shaped portion. Instead of or in addition to a shaped portion (such as a U-shaped portion), in some embodiments various clips, straps, snap-fit mechanisms, friction-fit mechanisms, or fasteners can be used to retain the fluid delivery device on the base receiver 202.

In the example of FIG. 2, a U-shaped portion 208 can be positioned along any length of the base receiver 202. As illustrated, the U-shaped portion 208 can be positioned at or near the middle of the base receiver 202. Alternatively, the U-shaped portion 208 can be positioned at or near a distal end 218 of the base receiver 202. In other examples, the U-shaped portion 208 can be positioned at or near a proximal end 220 of the base receiver 202. In some embodiments, the base receiver 202 can include one U-shaped portion 208, however more than one U-shaped portions are contemplated herein. For example, the base receiver 202 can include one, two, three, or four U-shaped portions. In some embodiments, the base receiver 202 can further include a flange receiving slot 210. The flange receiving slot 210 can be positioned at the proximal end 220 of the base receiver 202. The flange receiving slot 210 can be configured to receive a flange of the fluid delivery device arranged near a proximal end of the barrel.

The plunger holder 204 can be configured to engage with a plunger of the fluid delivery device. The plunger holder 204 can be positioned proximal to the flange receiving slot 210. The plunger holder 204 can be slidably connected to the base receiver 202. The plunger holder 204 can include a plunger flange receiving slot 212 configured to engage with a plunger of the fluid delivery device. In such embodiments, the plunger holder 204 can overlap with a proximal end of the plunger of the fluid delivery device. However, it will be appreciated that various mechanisms are contemplated herein to attach the plunger holder 204 to a plunger of a fluid delivery device.

The movement sensor can be configured to detect a movement speed of the plunger holder 204 relative to the base receiver 202. The movement sensor can be positioned along a length of the base receiver 202. In some embodiments, the flow rate measurement device 200 can be configured to generate a signal related to the movement speed of the plunger holder 204 relative to the base receiver 202. In some embodiments, the flow rate measurement device 200 can be configured to calculate a fluid flow rate based on the movement speed of the plunger holder 204. For example, the fluid flow rate can be calculated using a control circuit (described further below) disposed within the base receiver 202, the control circuit can be configured to receive the signal from the movement sensor 206 and calculate the fluid flow rate based on the signal. For example, the control circuitry can receive a signal reflecting the linear speed of the plunger holder 204 relative to the base receiver 202 and multiply the linear speed by a factor which is equal to the area of the plunger of the fluid delivery device in contact with the fluid contained within the fluid delivery device. In some embodiments, the multiplication factor can be preprogrammed into the control circuit memory.

In this example, the flow rate measurement device 200 also includes a side slot 214 and a display screen 216. The side slot 214 and the display screen 216 can both be positioned on the distal end 218 of the flow rate measurement device 200. The side slot can be configured to allow insertion of a fluid delivery device barrel into the base receiver 202 while connected to a fluid tube. E.g., the side slot 214 can allow for passage of a fluid tube that connects to the fluid delivery device. The side slot 214 will be discussed in further detail below with respect to FIG. 6. In some embodiments, the display screen 216 can be tiltable to allow a clinician or device operator to easily manipulate the display screen 216 to read the displayed information. The display screen 216 can be in electronic communication the control circuitry (described below) such that the display screen can be configured to display information related to the movement speed of the plunger holder 204 to the base receiver 202.

Additionally, or alternatively, the display screen 216 can be configured to display information related to the calculated fluid flow rate. In some embodiments, the display screen 216 can include a color bar on one or more edges of the display screen 216. The color bar can serve to provide a visual indication of the fluid flow rate to the clinician or device operator. For example, the color bar can display a green color if the clinician or device operator is within an acceptable or target range for the fluid flow rate. Alternatively, the color bar can display a red color if the clinician or device operator is outside of the acceptable or target range for the fluid flow rate. It can be noted that other colors are contemplated herein. For example, the color bar can display a yellow color if the clinician or device operator is minimally outside of the acceptable or target range for the fluid flow rate. In some embodiments, the display screen 216 can display the rounded-off value of the calculated fluid flow rate in real time as the clinician or device operator is manipulating the fluid delivery device.

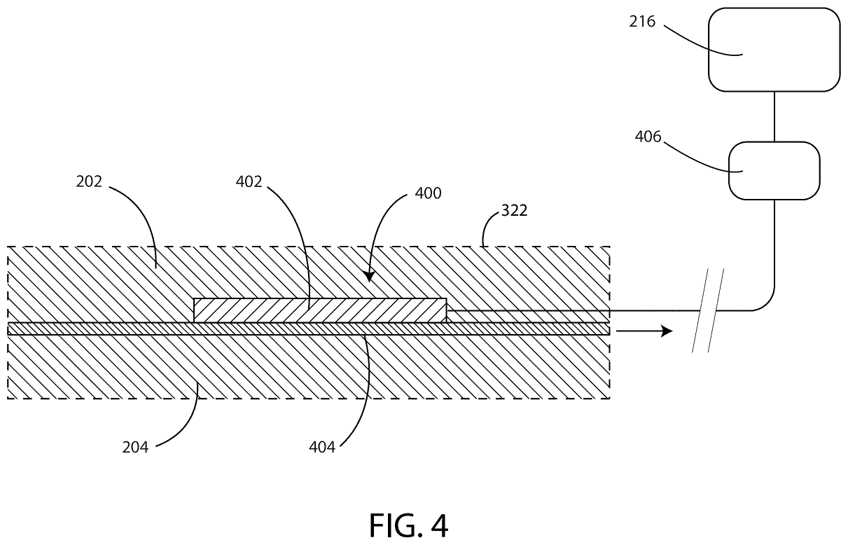

Side views and top views of the flow rate measurement device 200 are provided herein. Referring now to FIG. 3, a side elevation view of a flow rate measurement device is shown in accordance with various embodiments herein. FIG. 3 shows a flow rate measurement device 200, as before, including a base receiver 202, and a plunger holder 204. FIG. 3 also illustrates a portion 322 where the movement sensor (described below in FIG. 4) can be located some embodiments. The base receiver 202 includes a U-shaped portion 208, a flange receiving slot 210, a side slot 214, and a display screen 216. The plunger holder 204 includes a plunger flange receiving slot (not shown in this view). The plunger holder 204 can include an extension arm 320 that at least partially engages with (and can fit within in some embodiments) the base receiver 202 and can slide with respect to the same as a plunger for a syringe or other fluid delivery device is withdrawn and/or depressed.

In operation, the plunger holder 204 can slide back and forth with respect to the base receiver 202 between a fully depressed position 304 and a fully withdrawn position 306, such as by manual manipulation of the device user. For context, FIG. 3 also shows a current position 302 of the plunger holder 204 with respect to the base receiver 202. Movement of the plunger holder 204 (and/or aspects of the same such as distance and speed) with respect to the base receiver 202 can be sensed using the movement sensor.

Referring now to FIG. 4, a schematic sectional view is shown of a portion 322 of the flow rate measurement device of FIG. 3. The portion 322 shown includes base receiver 202 and plunger holder 204. The portion 322 shown also includes movement sensor 400. The movement sensor 400 can take various forms. In some embodiments, the movement sensor 400 can include a linear position sensor. The linear position sensor can include at least one of a linear encoder, an inductive position sensor, a linear variable differential transformer (LVDT), an ultrasonic position sensor, a laser position sensor, a Hall Effect sensor, a linear potentiometer, a magnetic linear position sensor, and a magnetostrictive linear position sensor. The movement sensor 400 can be in signal communication with a control circuitry 406.

In the embodiments shown in FIG. 4, the movement sensor 400 can include components such as a sensor or reader 402 and a scale 404. In some embodiments, other components can be used. For example, in some embodiments one or more membrane potentiometers can be used as the movement sensor. The membrane potentiometer can include a collector circuit, a resistor circuit, and a circuit spacer separating the two. A wiper (non-conductive mechanism) can be positioned on plunger holder 204 and the membrane potentiometer can be positioned on the base receiver 202 (such components can also be reversed in some embodiments). Movement of the plunger holder 204 relative to the base receiver 202 can cause the wiper to depress the collector circuit and the resistor circuit together creating a potentiometric output which can be provided to the control circuitry 406 to sense movement.

The control circuitry 406 can include various components to execute operations herein including one or more of a microprocessor, an application specific integrated circuit (ASIC), memory circuit (such as random access memory (RAM), read only memory (ROM)) and/or Electrically Erasable ROM (EEROM/Flash), recorder circuitry, controller circuit, a telemetry circuit, a power supply circuit (including, for example, a battery), a timing circuit, and, a recharging circuit, amongst others components. It will be appreciated that the device and/or components thereof such as the control circuitry can also function on an external power source or alternate power source (solar cell, rechargeable batteries, and the like).

The control circuitry 406 can perform calculations herein, such as calculating a plunger depression speed based on signals from the movement sensor 400 and/or calculating a flow rate for a syringe or other fluid delivery device that is inserted into the flow rate measurement device based on signals from the movement sensor 400 (wherein a calculated flow rate can be equal to the speed of plunger depression times the cross-sectional area of the barrel of the syringe or other fluid delivery device. The control circuitry 406 can also compare a calculated speed of plunger depression and/or flow rate and compare the same with predetermined target bounds. The control circuitry 406 can provide outputs based, in part, on whether current calculated speed of plunger depression and/or flow rates are within such bounds. The control circuitry 406 can also include a visual output circuit and can be in signal communication with display screen 216 in order to display information (such as a calculated speed of plunger depression, a calculated flow rate, an indication of whether such measures are within target bounds, and the like) to a device user. In some embodiments, such information can also be provided to another system or device using wired or wireless means via a telemetry circuit or other communication hardware.

The display screen 216 can be integrated with other components herein or can be separate or consist of any other external device display for displaying the data herein. The data can be relayed either with a wired connection or a wireless connection (such as using a BLE module in the device or separate display unit or another wireless protocol), using an IP protocol (via WIFI, a wired connection, or a cloud-based system) or any mobile device. The display screen can be powered externally or by the flow rate measurement device.

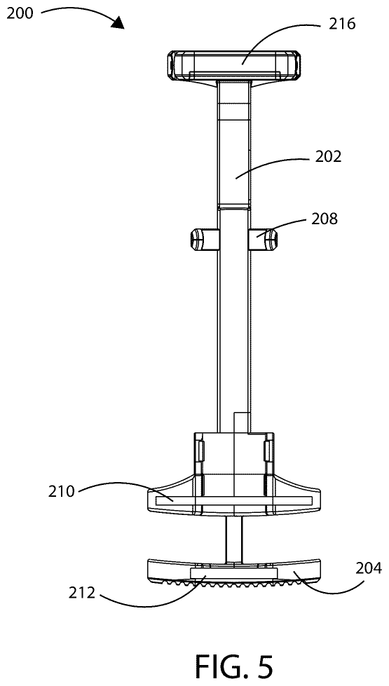

Referring now to FIG. 5, a top plan view of a flow rate measurement device is shown in accordance with various embodiments herein. FIG. 5 shows a flow rate measurement device 200, as before, including a base receiver 202, and a plunger holder 204. The base receiver 202 includes a U-shaped portion 208, a flange receiving slot 210, and a display screen 216. It is noted the side slot is not shown in this view. The plunger holder 204 includes a plunger flange receiving slot 212.

Referring now to FIG. 6, a perspective view of a fluid delivery device 600 being inserted into a flow rate measurement device is shown in accordance with various embodiments herein. In some embodiments, the fluid delivery device 600 can specifically include a syringe. A clinician or device operator can attach a fluid tube 602 to the fluid delivery device 600 and, in some cases, perform various operations such as system priming. The fluid tube 602 can then be inserted into a side slot 214 of the flow rate measurement device 200. In some embodiments, the fluid tube 602 can be inserted via a snap fit mechanism.

Once the fluid tube 602 is secured within the side slot 214, the clinician or device operator can engage a barrel 604, a flange 606, and a plunger 608 of the fluid delivery device 600 with the flow rate measurement device 200. For example, the barrel 604 can be engaged with a U-shaped portion or other connection portion of the base receiver 202. The U-shaped portion or connection portion can be configured to engage at least partially engage with the barrel 604 of the fluid delivery device 600 or another part thereof. In some embodiments, the base receiver 202 can retain the barrel 604 of the fluid delivery device 600 via a snap-fit mechanism. In other embodiments, the base receiver 202 can retain the barrel 604 of the fluid delivery device 600 via a pressure-fit mechanism. It will be appreciated that in some embodiments the barrel 604 and the base receiver 202 can be physically integrated as a single component, such as in the case of a single use device. Similarly, the plunger 608 and the plunger holder 204 can be physically integrated as a single component.

Further, the flange 606 can be engaged with the flange receiving slot 210 of the base receiver 202. The flange receiving slot 210 can be configured to at least partially engage with the flange 606 of the fluid delivery device 600 and retain the same. In some embodiments, the flange receiving slot 210 can retain the flange 606 of the fluid delivery device 600 via a snap-fit mechanism.

Lastly, the plunger 608 can be engaged with the plunger flange receiving slot 212 of the plunger holder 204. The plunger flange receiving slot 212 can be configured to at least partially engage with the plunger 608 of the fluid delivery device 600. In some embodiments, the plunger holder 204 can retain the plunger 608 of the fluid delivery device 600 via a snap-fit or a friction-fit mechanism.

In some embodiments, the barrel 604, the flange 606, and the plunger 608 can be engaged with the flow rate measurement device 200 simultaneously. In other embodiments, one or more of the barrel 604, the flange 606, and the plunger 608 can be engaged in a stepwise sequential approach.

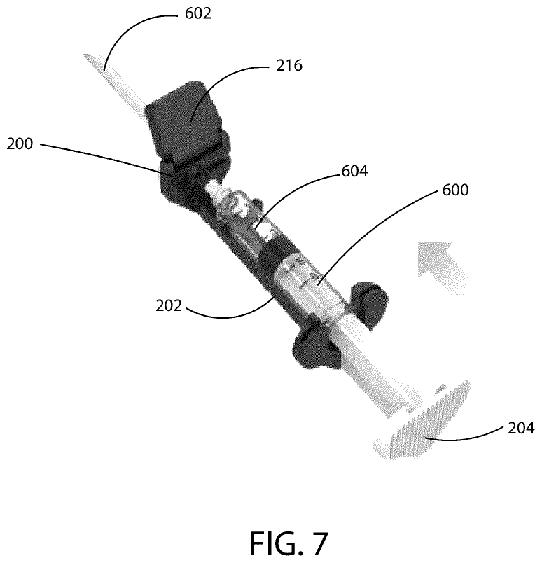

Referring now to FIG. 7, a schematic view of a fluid delivery device engaged with a flow rate measurement device is shown in accordance with various embodiments herein. In operation, a clinician or device operator can pull a plunger holder 204 of the flow rate measurement device 200 which will also move the plunger of the fluid delivery device which can cause a fluid to flow from a saline supply reservoir (not shown), through a fluid tube 602, and into a barrel 604 of a fluid delivery device 600.

After the fluid delivery device 600 is filled with fluid, the clinician or device operator can begin to depress the plunger holder 204 of the flow rate measurement device 200 causing fluid within the barrel 604 to be expelled from the fluid delivery device 600. As fluid is expelled, a movement sensor of the flow rate measurement device 200 detects a movement speed of the plunger holder 204 relative to the base receiver 202 and generates a signal related to the movement speed. That signal can then be converted into a fluid flow rate displayed on a display screen 216 of the fluid flow rate measurement device 200. The clinician or device operator can observe the fluid flow rate in real time while the plunger holder 204 is being depressed and adjust his/her depression speed as desired.

### Methods

Many different methods are contemplated herein, including, but not limited to, methods of making, methods of using, methods of measuring a fluid flow rate, and the like. Aspects of system/device operation described elsewhere herein can be performed as operations of one or more methods in accordance with various embodiments herein.

In various embodiments, operations described herein and method steps can be performed as part of a computer-implemented method executed by one or more processors of one or more computing devices. In various embodiments, operations described herein and method steps can be implemented instructions stored on a non-transitory, computer-readable medium that, when executed by one or more processors, cause a system to execute the operations and/or steps.

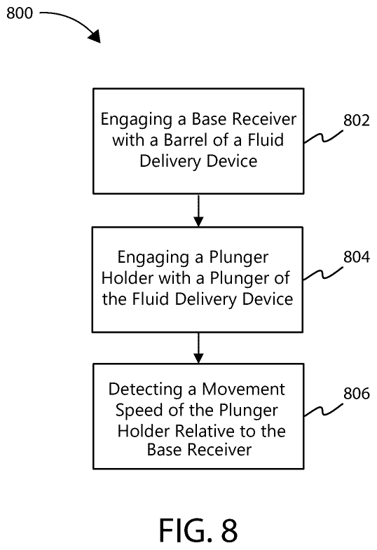

In an embodiment, a method of measuring fluid flow rate of a cancer therapy system is included. Referring now to FIG. 8, a flowchart of operations of a method is shown in accordance with various embodiments herein. The method of measuring fluid flow rate of a cancer therapy system 800 includes engaging a base receiver with a barrel of a fluid delivery device 802. The method of measuring fluid flow rate of a cancer therapy system also includes engaging a plunger holder with a plunger of the fluid delivery device 804. The method of measuring fluid flow rate of a cancer therapy system also includes detecting and/or calculating a movement speed of the plunger holder relative to the base receiver 806. In some embodiments, detecting a movement speed of the plunger holder relative to the base receiver can be performed using a movement sensor.

In various embodiments, the method can further include calculating a fluid flow rate based on a movement speed of the plunger holder. Calculations can be performed as described elsewhere herein. In various embodiments, the method can further include displaying the calculating fluid flow rate for a system user. In various embodiments, the method can further include generating a signal related to the movement speed of the plunger holder relative to the base receiver.

It should be noted that, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

As used herein, the recitation of numerical ranges by endpoints shall include all numbers subsumed within that range (e.g., 2 to 8 includes 2.1, 2.8, 5.3, 7, etc.).

The headings used herein are provided for consistency with suggestions under 37 CFR 1.77 or otherwise to provide organizational cues. These headings shall not be viewed to limit or characterize the invention(s) set out in any claims that may issue from this disclosure. As an example, although the headings refer to a “Field,” such claims should not be limited by the language chosen under this heading to describe the so-called technical field. Further, a description of a technology in the “Background” is not an admission that technology is prior art to any invention(s) in this disclosure. Neither is the “Summary” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

1. A flow rate measurement device for cancer therapy comprising:
a base receiver, wherein the base receiver is configured to engage with a barrel of a fluid delivery device;
a plunger holder, wherein the plunger holder is configured to engage with a plunger of the fluid delivery device; and
a movement sensor, wherein the movement sensor is configured to detect a movement speed of the plunger holder relative to the base receiver; and
wherein the flow rate measurement device for cancer therapy is configured to generate a signal related to the movement speed of the plunger holder relative to the base receiver.

2. The flow rate measurement device for cancer therapy of claim 1, wherein the flow rate measurement device for cancer therapy is configured to calculate a fluid flow rate based on a movement speed of the plunger holder.

3. The flow rate measurement device for cancer therapy of claim 1, further comprising a control circuit, wherein the control circuit is configured to receive a signal from the movement sensor and calculate a fluid flow rate based on the same.

4. The flow rate measurement device for cancer therapy of claim 1, the fluid delivery device comprising a syringe.

5. The flow rate measurement device for cancer therapy of claim 1, the movement sensor comprising a linear position sensor.

6. The flow rate measurement device for cancer therapy of claim 5, the linear position sensor comprising at least one selected from the group consisting of a linear encoder, an inductive position sensor, a linear variable differential transformer (LVDT), an ultrasonic position sensor, a laser position sensor, a Hall Effect sensor, a linear potentiometer, a magnetic linear position sensor, and a magnetostrictive linear position sensor.

7. The flow rate measurement device for cancer therapy of claim 1, the base receiver comprising a U-shaped portion.

8. The flow rate measurement device for cancer therapy of claim 7, wherein the U-shaped portion is configured to at least partially engage with the barrel of the fluid delivery device.

9. The flow rate measurement device for cancer therapy of claim 1, the base receiver defining a side slot, wherein the side slot is configured to allow insertion of a fluid delivery device barrel connected to a fluid tube into the base receiver.

10. The flow rate measurement device for cancer therapy of claim 1, the base receiver defining a flange receiving slot, wherein the flange receiving slot is configured to receive a flange of the fluid delivery device arranged near a proximal end of the barrel.

11. The flow rate measurement device for cancer therapy of claim 1, wherein the base receiver is configured to retain a barrel of a fluid delivery device via a snap-fit or pressure-fit mechanism.

12. The flow rate measurement device for cancer therapy of claim 1, further comprising a display screen, wherein the display screen is configured to display information related to the movement speed of the plunger holder relative to the base receiver and/or a calculated fluid flow rate.

13. The flow rate measurement device for cancer therapy of claim 12, wherein the display screen is configured to display a particular color if a calculated fluid flow rate falls within a predetermined range.

14. The flow rate measurement device for cancer therapy of claim 1, wherein the plunger holder overlaps with a proximal end of the plunger of the fluid delivery device.

15. A method of measuring fluid flow rate of a cancer therapy system comprising:
engaging a base receiver with a barrel of a fluid delivery device;
engaging a plunger holder with a plunger of the fluid delivery device; and
detecting a movement speed of the plunger holder relative to the base receiver.

16. The method of claim 15, further comprising calculating a fluid flow rate based on a movement speed of the plunger holder.

17. The method of claim 16, further comprising displaying the calculated fluid flow rate for a system user.

18. The method of claim 15, further comprising generating a signal related to the movement speed of the plunger holder relative to the base receiver.

19. The method of claim 15, wherein detecting a movement speed of the plunger holder relative to the base receiver is performed using a movement sensor.

20. The method of claim 15, the fluid delivery device comprising a syringe.
